(19) United States
(12) Patent Application Publication
(10) Pub. No.: US 2014/0221945 A1
(43) Pub. Date: Aug. 7, 2014

(54) MULTI-DOSE DISPENSER AND APPLICATOR FOR TOPICAL MEDICATIONS

(71) Applicant: Medisca Pharmaceutique, Inc., Saint-Laurent (CA)

(72) Inventors: Antonio Dos Santos, Saint-Laurent (CA); Panagiota Danopoulos, Saint-Laurent (CA); Hans Lickliter, Canyon Country, CA (US); Sarah Nyat Peng Wong, Montreal (CA)

(73) Assignee: Medisca Pharmaceutique, Inc., Saint-Laurent (CA)

(21) Appl. No.: 14/172,170

(22) Filed: Feb. 4, 2014

Related U.S. Application Data
(60) Provisional application No. 61/760,263, filed on Feb. 4, 2013.

(51) Int. Cl. A61M 35/00

(52) U.S. Cl. CPC A61M 35/003
USPC 604/311

(57) ABSTRACT

A multi-dose, metered dispenser and applicator for topical medications includes inner and outer cylinders and a plurality of expanding bases. These bases form part of a telescopic piston drive system that advances the main piston through the inner cylinder, in accordance with a selected dosage, to dispense a metered amount of the topical medication. The dosage may be selected by a switch located on an outside of the outer cylinder, and dispensed by rotating the inner cylinder relative to the outer cylinder. A tactile and audible feedback mechanism informs the user that the desired dosage of medication has been dispensed.

This application claims the benefit of U.S. Provisional Application No. 61/760,263, filed on Feb. 4, 2013. The entire disclosure of the above application is hereby incorporated herein by reference.

### FIELD OF THE INVENTION

The present disclosure relates to a dispenser and applicator for a topical medication and, more particularly, a dispenser and applicator for selectively dispensing different metered doses of a topical medication.

### BACKGROUND OF THE INVENTION

Topical dispensers known in the art are disclosed, for example, in U.S. Pat. No. 7,213,994 and U.S. Pat. No. 7,303,348, both to Phipps et al. The known topical dispensers are for metered dosing of cream-based medicines. The dispensers include a barrel, a base having a threaded rod extending therefrom, a riser having at least one flexible seal which engages the barrel, and an applicator cap having apertures therein for spreading dispensed cream onto a user's skin. The user positively knows when a metered amount of cream has been dispensed by tactile and audible feedback.

There is a continuing need for an improved multi-dose dispenser for topical application of medications. Desirably, the topical dispenser facilitates a selection and application by the user of multiple different doses of the medications.

### SUMMARY OF THE INVENTION

In concordance with the instant disclosure, an improved multi-dose dispenser for topical application of medications, which facilitates a selection by the user of multiple different doses of the medications, is surprisingly discovered.

The present disclosure includes a multi-dose dispenser and applicator for topical medications. In an exemplary embodiment, the multi-dose dispenser and applicator has a cylindrical body, and includes an inner cylinder top ring, an inner cylinder, a cylinder sprocket, an outer cylinder, a safety lid, a switch that may consist of a top button and a main button, a main cap, a main piston, an expanding plunger base, and a plurality of expanding bases. The number of expanding bases is dependent on the volume size of the multi-dose dispenser and applicator. The dispensing function in the multi-dose dispenser and applicator involves a telescoping actuation, as opposed to actuation by a drive screw. The telescopic actuation system allows for facilitated filling of the topical medication into the inner cylinder, and enables a multi-dosage capability.

In a particular embodiment, the multi-dose dispenser and applicator for topical medications may be provided in a model based on volume size, for example, a model configured to contain a medication in a total amount of 65 mL, 35 mL, or 15 mL. The device is generally cylindrical in shape, consisting of an inner cylinder top ring, an inner cylinder, a cylinder sprocket, an outer cylinder, a safety lid, a switch that may consist of a top button and a main button, a main cap, a main piston, an expanding plunger base, and several expanding bases of which the number is dependent on the volume size of the particular device model. The device may also include a view window that consists of a clear plastic strip down the length of the cylinder. The view window allows for viewing of the content inside the dispenser.

In a filling operation, the inner cylinder can be filled with a topical medication to the fill-line which indicates the total containment volume of the particular device model. The main cap can then be snapped onto the top of the inner cylinder by administering a minimal force from the top downward. Once snapped in place, the main cap cannot be removed to prevent tampering or contamination of the stored content.

In an end-use operation, the safety lid can be removed by pressing the two interlock tabs, which are on opposite sides of the inner cylinder top ring, simultaneously while lifting the safety lid upward. By vertically sliding the switch up or down, the device can be set to one of three dosage choices, for example: 0.10 mL, 0.25 mL, or 0.50 mL. The expanding bases comprise a threaded telescopic piston drive system which advances the main piston up the inner cylinder when the inner cylinder top ring is twisted one of clockwise and counter clockwise. The completion of each turn is confirmed by the tactile and acoustic feedback of a clicking action produced by a ratchet mechanism between the switch and inner cylinder. This ratchet system also prevents misuse or incorrect dosing by preventing the inner cylinder top ring from turning in the wrong direction. The degree by which the cylinder is twisted will vary according to the dosage setting. At each turn, the piston pushes out the allotted dosage through the hole in the main cap. The topical content can then be applied by simply rubbing the top of the device (i.e., the main cap as the safety lid is removed) onto the area to be treated.

In one embodiment, a dispenser for a topical medication includes a ratchet system and a piston drive system. The ratchet system permits a user to selectively provide one of a plurality of measured doses of the topical medication using at least one of a tactile feedback and an acoustic feedback. The ratchet system includes an outer cylinder having a switch for selecting one of the plurality of measured doses of the topical medication. An inner cylinder is disposed within of the outer cylinder, and has a plurality of gears that cooperate with the switch to provide at least one of the tactile feedback and the acoustic feedback during a rotation of the inner cylinder relative to the outer cylinder. The inner cylinder is configured to contain the topical medication. The piston drive system has a piston disposed within the inner cylinder, which is configured to advance along an axial length of the inner cylinder with the rotation of the inner cylinder relative to the outer cylinder. The piston drive system is configured to dispense the topical medication from the inner cylinder, in an amount corresponding to the one of the plurality of measured doses selected, by the rotation of the inner cylinder relative to the outer cylinder. A completion of the rotation is confirmed by the at least one of the tactile feedback and the acoustic feedback.

In another embodiment, the piston drive system of the dispenser includes a plurality of expanding bases that are configured to advance the piston along the axial length of the inner cylinder with the rotation of the inner cylinder relative to the outer cylinder.

In a further embodiment, the dispenser includes further a main cap disposed on an open end of the inner cylinder. The main cap has at least one hole configured to dispense the topical medication from the inner cylinder. The dispenser also has an inner cylinder top ring disposed over the outer cylinder and coupled with the inner cylinder. The inner cylinder top ring permits the rotation of the inner cylinder relative to the outer cylinder. The dispenser also includes a safety lid selectively coupled with the inner cylinder top ring. Optionally, the dispenser may include a clear view window formed in the outer cylinder.

### DRAWINGS

The above, as well as other advantages of the present disclosure, will become readily apparent to those skilled in the art from the following detailed description, particularly when considered in the light of the drawings described herein.

FIG. 1 is a perspective view showing a topical dispenser according to one embodiment of the present disclosure;

FIGS. 2A-2C are a side elevational view, a front elevational view, and rear elevational view, respectively, of the topical dispenser illustrated in FIG. 1, with an optional view window further shown in FIG. 2C;

FIG. 3 is a front elevational cross-sectional view of the topical dispenser taken along section line 3-3 in FIG. 1;

FIGS. 4A-4B are a top exploded perspective view and a bottom exploded perspective view, respectively, of the topical dispenser illustrated in FIG. 1, a piston drive system of the topical dispenser illustrated in an exploded view in FIG. 4A and the piston drive system illustrated in an assembled view in FIG. 4B;

FIG. 5 is a front elevational view of the piston drive system for the topical dispenser illustrated in FIG. 4B, the piston drive system shown in a collapsed position;

FIG. 6 is a cross-sectional elevational view of the assembled telescopic drive system taken along section line 6-6 in FIG. 5;

FIG. 7 is an exploded perspective view of the telescopic drive system illustrated in FIG. 6; and

FIGS. 8A-8B are a front exploded perspective view and a rear exploded perspective view, respectively, of a ratchet system for the topical dispenser illustrated in FIG. 1.

### DETAILED DESCRIPTION OF THE INVENTION

The following detailed description and appended drawings describe and illustrate various embodiments of the invention. The description and drawings serve to enable one skilled in the art to make and use the invention, and are not intended to limit the scope of the invention in any manner. In respect of the methods disclosed, the order of the steps presented is exemplary in nature, and thus, are not necessary or critical unless otherwise stated.

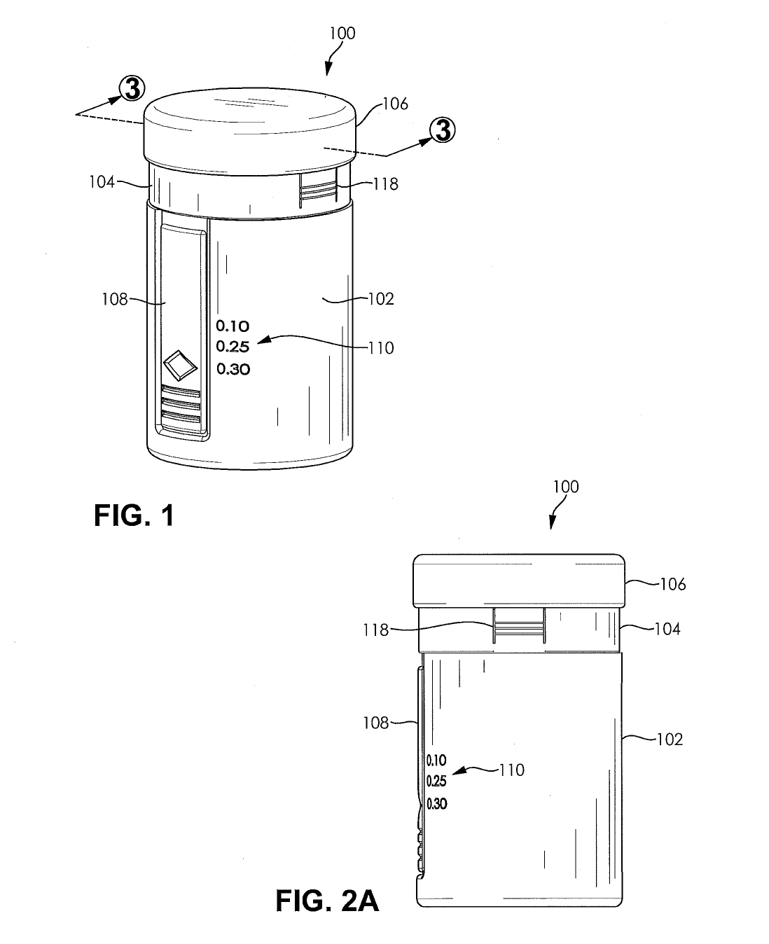

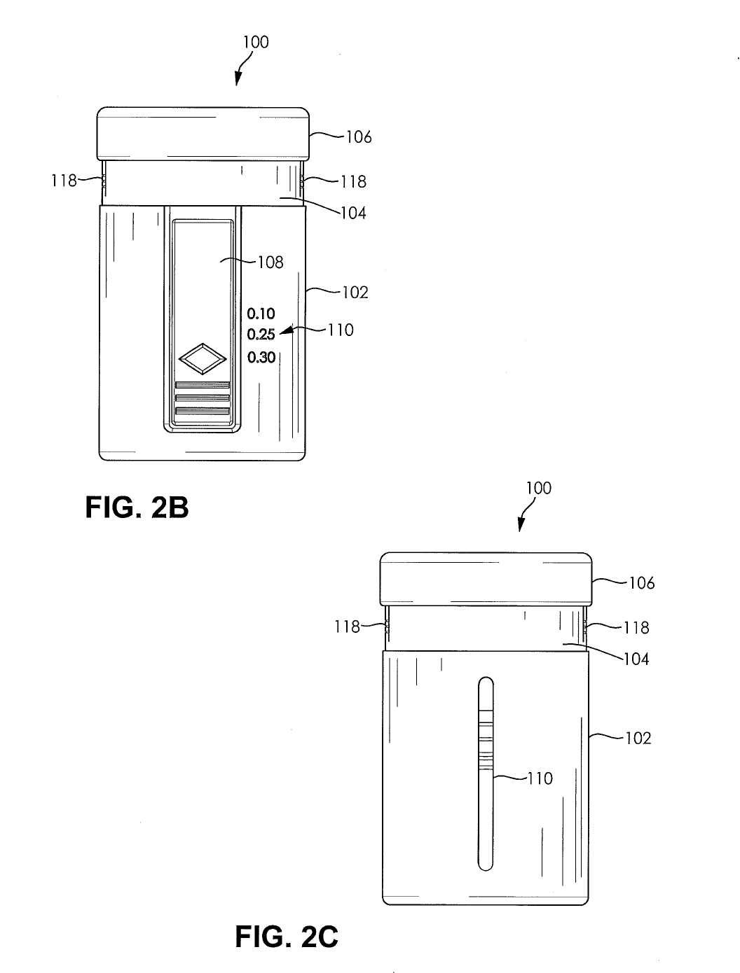

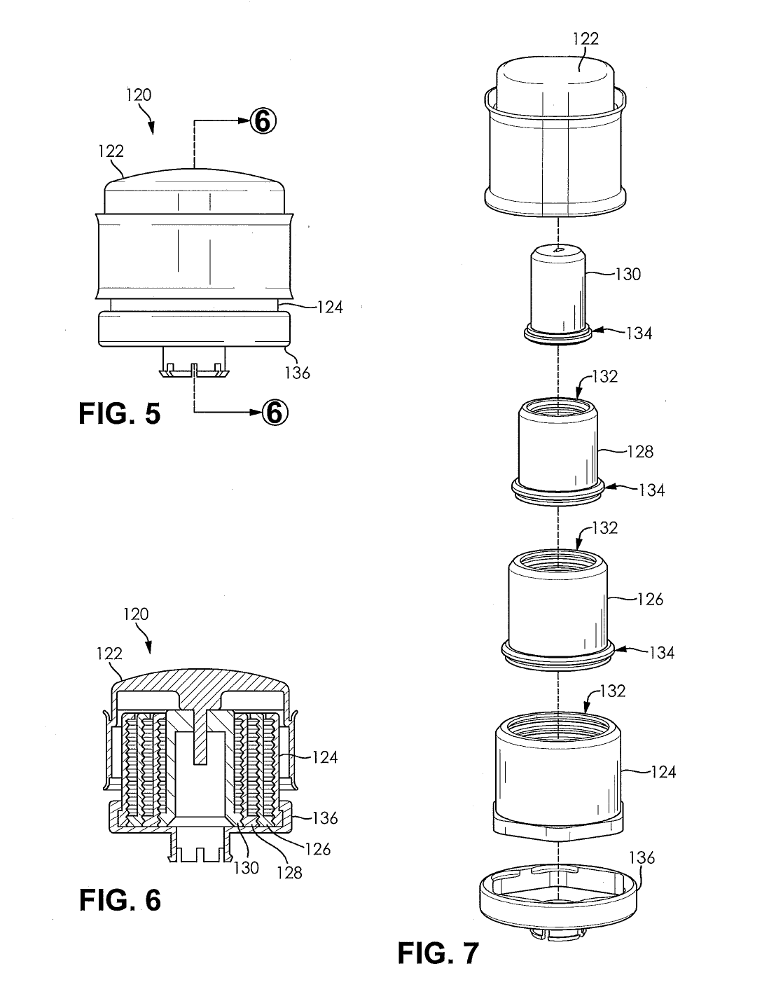

FIGS. 1-8 of the present disclosure show a multi-dose dispenser 100 for a topical medication (not shown). The multi-dose dispenser 100 permits a user to selectively provide one of a plurality of measured doses of the topical medication using at least one of a tactile feedback and an acoustic feedback. As shown in FIGS. 1-2C, the multi-dose dispenser 100 may have a main body including an outer cylinder 102, an inner cylinder top ring 104, a safety lid 106, and a switch 108. The outer cylinder 102 may have a plurality of markings 110, which are indicative of the plurality of measured doses of the topical medication that may be selected with the switch 108. Although three measured doses are shown, for purposes of simplicity, one of ordinary skill in the art may manufacturer the multi-dose dispenser 100 with the ability to select any number of measured doses, as desired.

Optionally, the multi-dose dispenser 100 may include a clear view window 110 disposed along a length of the outer cylinder 102, for example, as shown in FIG. 2C. Where the window 110 is used, the inner cylinder 116 may be formed from a transparent material. The clear view window 110 permits a viewing of topical medication within the multi-dose dispenser 100.

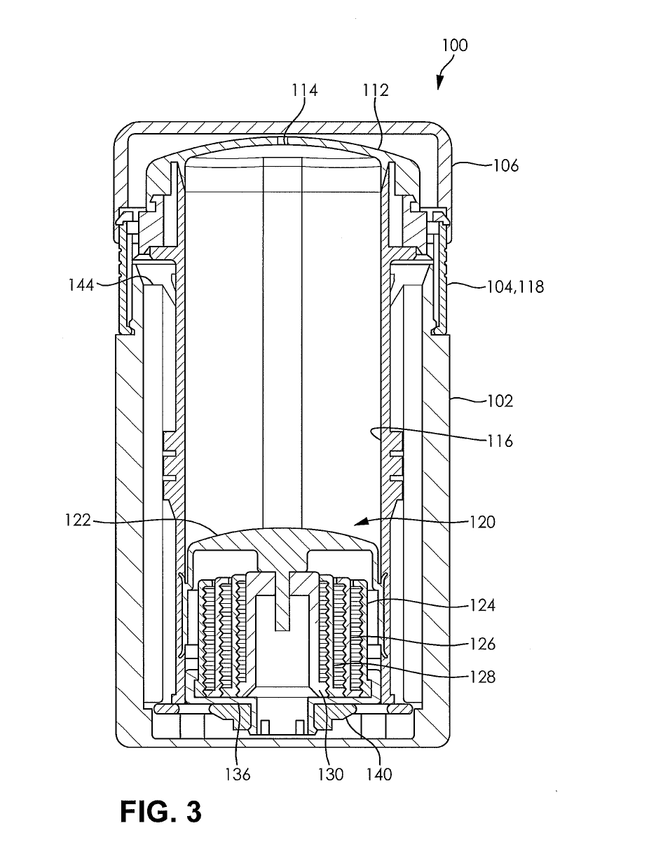

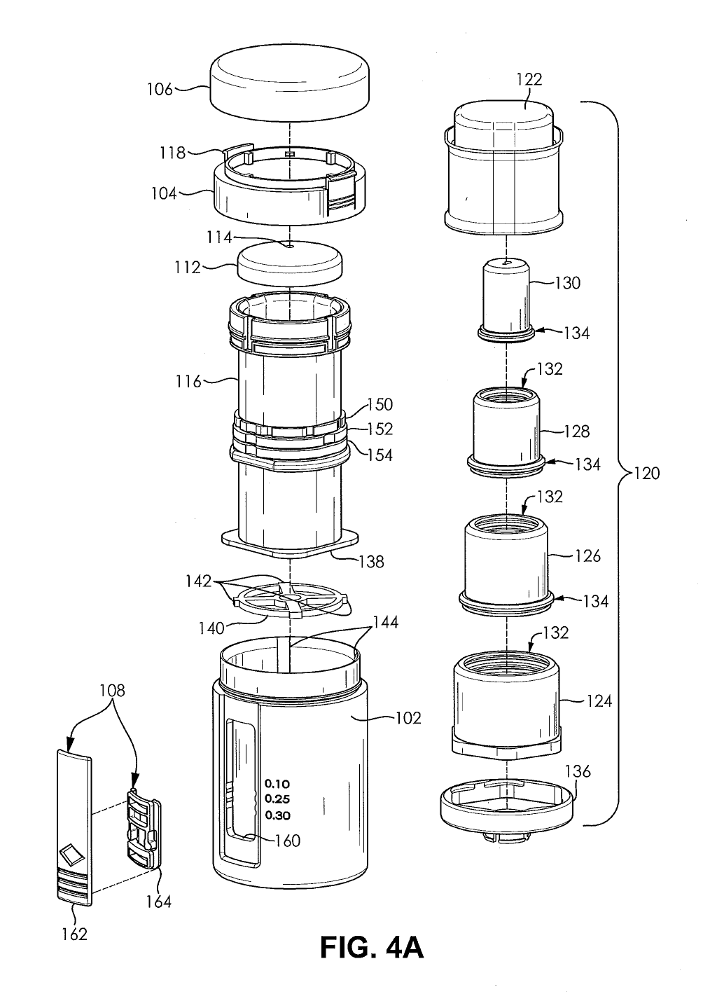

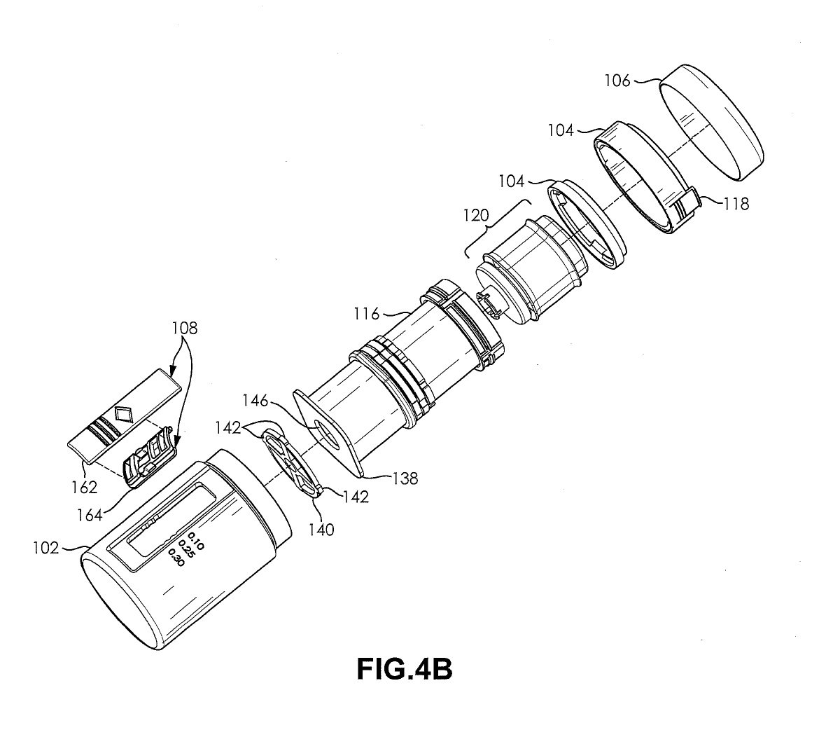

As shown in FIGS. 3 to 4B, the multi-dose dispenser 100 includes a main cap 112. The main cap 112 has at least one hole 114 configured to dispense the topical medication. The main cap 112 is disposed on an open end of an inner cylinder 116, and has an upper surface that facilitates an application of the dispensed topical medication. The inner cylinder 116 is disposed within the outer cylinder 102, and is configured to contain the topical medication before it is dispensed from the multi-dose dispenser 100.

The inner cylinder top ring 104 of the multi-dose dispenser 100 is disposed over the outer cylinder 102. The inner cylinder top ring 104 is also coupled with the inner cylinder 116. A rotation of the inner cylinder 116 relative to the outer cylinder 102 is permitted by holding one of the outer cylinder 102 and the inner cylinder top ring 104 stationary, while twisting the other of the outer cylinder 102 and the inner cylinder top ring 104. It should be appreciated that such an operation may be easily and accurately performed by a user of the multi-dose dispenser 100 according to the present disclosure.

The safety lid 106 of the multi-dose dispenser 100 may be selectively coupled with the inner cylinder top ring 104. For example, the inner cylinder top ring 104 may include a pair of interlocking tabs 118 that are disposed on opposite sides of the inner cylinder top ring 104. The interlocking tabs 118 may be depressed and then inserted into the safety lid, where they press against an inner surface of the safety lid 106. Each of the interlocking tabs 118 may further have a lip that cooperates with a ridge formed on an inner surface of the safety lid 106 to secure the inner cylinder top ring 104 with the safety lid 106. The safety lid 106 may subsequently be removed by again depressing the interlocking tabs 118, and pulling the safety lid from the multi-dose dispenser 100. Other means or fasteners for selectively coupling the safety lid 106 with the inner cylinder top ring 104 may also be employed, as desired.

The multi-dose dispenser 100 of the present disclosure further includes a piston drive system 120. As shown in FIGS. 3-7, the piston drive system 110 includes a piston 122 and a plurality of expanding bases 124, 126, 128, 130. The piston 122 and the expanding bases 124, 126, 128, 130 are disposed within the inner cylinder 116. The piston 122 has a shape that does not permit the piston 122 to rotate within the inner cylinder 116. A cross-sectional shape of the piston 122 may conform to a cross-sectional shape a cavity of the inner cylinder 116 within which the piston 122 is disclosed. As nonlimiting examples, a suitable cross-sectional shape for the piston 122 and the cavity of the inner cylinder 116, which militates against a rotation of the piston 122 within the cavity of the inner cylinder 116, may be one of substantially square and substantially rectangular. One of ordinary skill in the art may select other shapes for the piston 122 and the cavity of the inner cylinder 116 within the scope of the present disclosure.

The inner cylinder 116 is disposed within the outer cylinder 102. The piston drive system 120 is configured to advance the piston 122 along an axial length of the inner cylinder 116 with a rotation of the inner cylinder 116 relative to the outer cylinder 102. In this manner, the piston drive system 112 is also configured to dispense the topical medication from the inner cylinder 116, in an amount corresponding to the one of the plurality of measured doses selected.

As a nonlimiting example, the expanding bases 124, 126, 128, 130 may include a first expanding base 124, a second expanding base 126, a third expanding base 128, and a fourth expanding base 130. The first expanding base 124 has internal threads 132 and the second expanding base 126 has external threads 134. The internal threads 132 of the first expanding base 124 cooperate with the external threads 134 of the second expanding base 126. Likewise, the third expanding base 128 may have external threads 134 cooperating with the internal threads 132 of the second expanding base 126, and the fourth expanding base 130 may have the external threads 134 cooperating with the internal threads 132 of the third expanding base 128. The expanding bases 124, 126, 128, 130 thereby cooperate with one another, nested in series, with the piston 122 disposed on and supported by the fourth expanding base 130.

The piston drive system 120 further includes an expanding plunger base 136. The expanding plunger base 136 is disposed within the inner cylinder 116 adjacent a bottom wall 138 of the inner cylinder 116. The first expanding base 124 is coupled with the expanding plunger base 136. For example, the first expanding base 124 may conform to the shape of a recess formed in the expanding plunger base 136, and be nested within the recess of the expanding plunger base 136, such that the first expanding base 124 is rotated when the expanding plunger base 136 is rotated. Other means for coupling the first expanding base 124 with the expanding plunger base 136 may also be used within the scope of the present disclosure.

The piston drive system 120 may also include a cylinder sprocket 140. The cylinder sprocket 140 is disposed within the outer cylinder 102 adjacent the bottom wall 138 of the inner cylinder 116. The cylinder sprocket 140 has a plurality of teeth 142 that cooperate with a plurality of notches 144 formed on an inner surface of the outer cylinder 102. The cylinder sprocket 140 is coupled to the expanding plunger base 136 through an opening 146 formed in the bottom wall 138 of the inner cylinder 116. It should be appreciated that the rotation of the inner cylinder 116 relative to the outer cylinder 102 thereby results in corresponding rotations of the cylinder sprocket 140, the expanding plunger base 136, and the expanding bases 124, 126, 128, 130 of the piston drive system 120. Furthermore, a skilled artisan understands that, because the piston 122 is not permitted to rotate within the inner cylinder 116, expanding bases 124, 126, 128, 130 are caused to move from a collapsed position (shown in FIGS. 3 and 4B-6) to an expanded position. The piston 122 is thereby moved along the axial length of the multi-dose dispenser 100 to dispense the topical medication from the inner cylinder 116 of the multi-dose dispenser 100.

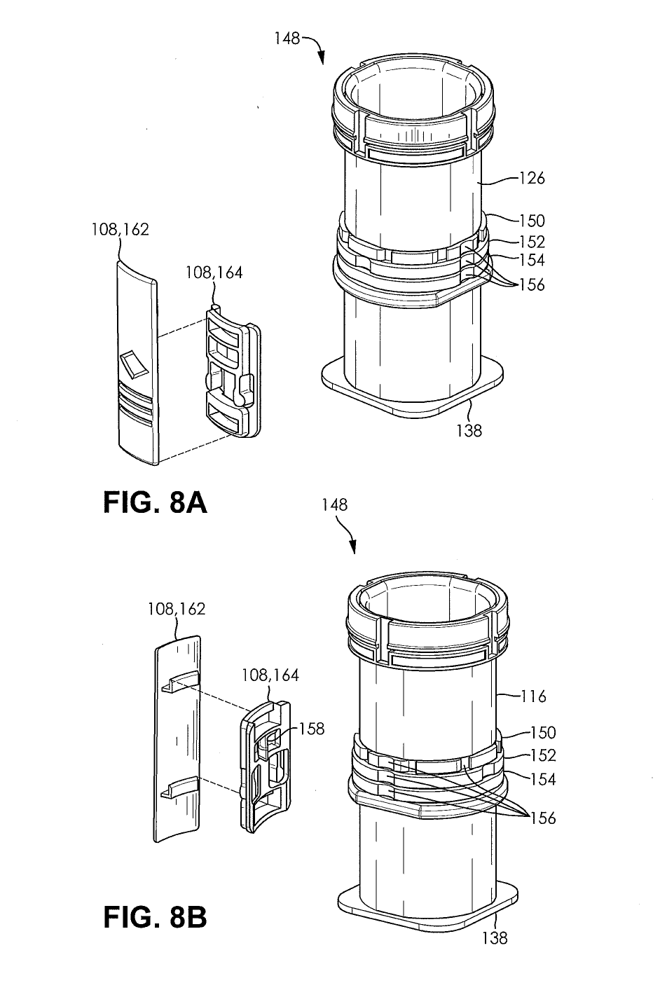

The multi-dose dispenser 100 of the present disclosure further includes a ratchet system 148. In operation, the ratchet system 148 causes at least one of the tactile feedback and the acoustic feedback, which is used to confirm when the measured doses of the topical medication have been dispensed. As shown in FIGS. 8A and 8B, the ratchet system includes the switch 108 of the outer cylinder 102 and the inner cylinder 116. The inner cylinder 116 may have a plurality of gears 150, 152, 154. The gears 150, 152, 154 cooperate with the switch 108 to provide at least one of the tactile feedback and the acoustic feedback during the rotation of the inner cylinder 116 relative to the outer cylinder 102.

The gears 150, 152, 154 are arranged adjacent one another on an outer surface of the inner cylinder 116, along the axial length of the inner cylinder 116. The gears 150, 152, 154 may be integral with the inner cylinder 116, or formed separately and then affixed to the inner cylinder 116, as desired. Each of the gears 150, 152, 154 has interruptions 156, in the form of either depressions or projections, disposed along the outer perimeters of the gears 150, 152, 154. The interruptions 156 of the gears 150, 152, 154 are configured to cooperate with a corresponding feature 158 on the switch 108, to create at least one of the tactile feedback and the acoustic feedback as the inner cylinder 116 is rotated relative to the outer cylinder 102. For example, the corresponding feature 158 may be a pawl. In particular, the interruptions 156 are configured to only allow rotational movement of the inner cylinder 116 relative to the outer cylinder 102 in one direction.

It should be appreciated that any number of gears 150, 152, 154 may be used within the scope of the present disclosure. In one example, the plurality of gears 150, 152, 154 may include a first gear 150, a second gear 152, and a third gear 154. The interruptions 156 of the first gear 150 are spaced apart a first interval, the interruptions 156 of the second gear 152 are spaced apart a second interval, and the interruptions 156 of the third gear 154 are spaced apart a third interval. As shown in FIGS. 8A and 8B, the third interval is greater in length than the second interval, and the second interval is greater in length than the first interval. A skilled artisan may select suitable lengths for the first, second, and third intervals, as desired.

In particular embodiments, the rotation of the inner cylinder 116 relative to the outer cylinder 102, along an entirety of the first interval, results in the topical medication being dispensed from the inner cylinder 116 by the piston 122 in an amount corresponding to a first measured dose. The rotation of the inner cylinder 116 relative to the outer cylinder 102, along an entirety of the second interval, results in the topical medication being dispensed from the inner cylinder 116 by the piston 122 in an amount corresponding to a second measured dose. The rotation of the inner cylinder 116 relative to the outer cylinder 102, along an entirety of the third interval, results in the topical medication being dispensed from the inner cylinder 116 by the piston 122 in an amount corresponding to a third measured dose. In a most particular embodiment, the third measured dose is greater than the second measured dose, and the second measured dose is greater than the first measured dose. One of ordinary skill in the art may select the amounts for the first measured dose, the second measured dose, and the third measured dose, as well as the corresponding lengths for the first interval, the second interval, and the third interval, respectively, as desired.

It should be appreciated that the switch 108 cooperates with only one of the gears at a time, during the rotation of the inner cylinder 116 relative to the outer cylinder 102. The switch 108 may be adjusted by the user to interact with the desired one of the gears 150, 152, 154. For example, as shown in FIGS. 4A to 4B, the switch 108 may be at least partly disposed through a slot 160 formed in the outer cylinder 102. In a particular example, the slot 160 is elongate and orientated vertically along the outer cylinder 102. In another particular example, the switch 108 may be two-part, including a top button 162 and a main button 164. The top button 162 is disposed on an outside of the outer cylinder 102, and may have a texture that facilitates an operation of the switch 108 by the user. The main button 164 may be disposed through the slot 160, and have the corresponding feature 158 formed thereon. A skilled artisan may select other types of switches 108 within the scope of the present disclosure.

In operation, the safety lid 106 of the multi-dose dispenser 100 may first be removed by pressing the two interlocking tabs 118, which are on opposite sides of the inner cylinder top ring 104, simultaneously while lifting the safety lid 106 upward. By vertically sliding the switch 108 up or down, the dispenser can be set to one of the three dosage choices: 0.10 mL, 0.25 mL, or 0.50 mL, as nonlimiting examples. The threaded telescopic piston drive system 120 with the expanding bases 124, 126, 128, 130 advances the piston 122 up the inner cylinder 116 when the inner cylinder top ring 104 is twisted, for example, one of counter clockwise and clockwise.

Advantageously, the completion of each turn is confirmed by one of the tactile feedback and the acoustic feedback, also known as a “clicking action”, produced by the ratchet system 148 between the switch 108 and one of the gears 150, 152, 154 of the inner cylinder 116. The ratchet system also militates against misuse or incorrect dosing by only permitting the rotation of the inner cylinder top ring 104 in one direction. At each turn, the piston 122 pushes out the allotted dosage through the hole 114 in the main cap 112. The topical medication can then be applied by simply rubbing the top of the multi-dose dispenser 100 (i.e., the main cap 112 after the safety lid 106 is removed) onto the area to be treated.

While certain representative embodiments and details have been shown for purposes of illustrating the invention, it will be apparent to those skilled in the art that various changes may be made without departing from the scope of the disclosure, which is further described in the following appended claims.

What is claimed is:

1. A multi-dose dispenser for a topical medication, comprising:
a ratchet system permitting a user to selectively provide one of a plurality of measured doses of the topical medication using at least one of a tactile feedback and an acoustic feedback, the ratchet system including an outer cylinder having a switch for selecting one of the plurality of measured doses of the topical medication, and an inner cylinder disposed within of the outer cylinder, the inner cylinder having a plurality of gears that cooperate with the switch to provide the at least one of the tactile feedback and the acoustic feedback during a rotation of the inner cylinder relative to the outer cylinder, the inner cylinder configured to contain the topical medication; and
a piston drive system having a piston disposed within the inner cylinder and configured to advance along an axial length of the inner cylinder with the rotation of the inner cylinder relative to the outer cylinder, the piston drive system configured to dispense the topical medication from the inner cylinder, in an amount corresponding to the one of the plurality of measured doses selected, by the rotation of the inner cylinder relative to the outer cylinder, a completion of the rotation confirmed by the at least one of the tactile feedback and the acoustic feedback.

2. The multi-dose dispenser of claim 1, wherein the plurality of gears are arranged adjacent one another on an outer surface of the inner cylinder along the axial length of the inner cylinder.

3. The multi-dose dispenser of claim 1, wherein the plurality of gears includes a first gear and a second gear.

4. The multi-dose dispenser of claim 3, wherein interruptions in the first gear are spaced apart a first interval, and interruptions of the second gear are spaced apart a second interval.

5. The multi-dose dispenser of claim 4, wherein the second interval is greater in length than the first interval.

6. The multi-dose dispenser of claim 5, wherein the rotation of the inner cylinder relative to the outer cylinder along an entirety of the first interval results in the topical medication being dispensed from the inner cylinder by the piston in an amount corresponding to a first measured dose, and the rotation of the inner cylinder relative to the outer cylinder along an entirety of the second interval results in the topical medication being dispensed from the inner cylinder by the piston in an amount corresponding to a second measured dose.

7. The multi-dose dispenser of claim 6, wherein the second measured dose is greater than the first measured dose.

8. The multi-dose dispenser of claim 7, wherein the switch includes a top button disposed on an outside of the outer cylinder and coupled to a main button disposed at least partly within the outer cylinder, the main button having a corresponding feature configured to cooperate with the interruptions of the gears to cause the at least one of the tactile feedback and the acoustic feedback.

9. The multi-dose dispenser of claim 8, wherein the switch is disposed through an elongate slot formed in the outer cylinder.

10. The multi-dose dispenser of claim 1, wherein the switch cooperates with only one of the gears during the rotation of the inner cylinder relative to the outer cylinder to cause the at least one of the tactile feedback and the acoustic feedback.

11. A multi-dose dispenser for a topical medication, comprising:
a ratchet system permitting a user to selectively provide one of a plurality of measured doses of the topical medication by at least one of a tactile feedback and an acoustic feedback, the ratchet system including an outer cylinder for selecting one of the plurality of measured doses of the topical medication and an inner cylinder disposed within the outer cylinder, the inner cylinder configured to contain the topical medication; and
a piston drive system having a piston disposed within the inner cylinder, the piston drive system including a plurality of expanding bases that are configured to advance the piston along an axial length of the inner cylinder with a rotation of the inner cylinder relative to the outer cylinder, the piston drive system configured to dispense the topical medication from the inner cylinder, in an amount corresponding to the one of the plurality of measured doses selected, by the rotation of the inner cylinder relative to the outer cylinder, a completion of the rotation confirmed by the at least one of the tactile feedback and the acoustic feedback.

12. The multi-dose dispenser of claim 11, wherein the expanding bases include a first expanding base and a second expanding base, the first expanding base having internal threads and the second expanding base having external threads, the internal threads of the first expanding base cooperating with the external threads of the second expanding base.

13. The multi-dose dispenser of claim 12, wherein the piston drive system includes an expanding plunger base disposed within the inner cylinder adjacent a bottom wall of the inner cylinder, the first expanding base coupled with the expanding plunger base.

14. The multi-dose dispenser of claim 13, wherein the piston drive system includes a cylinder sprocket disposed within the outer cylinder adjacent a bottom wall of the inner cylinder, the cylinder sprocket coupled to the expanding plunger base through an opening in the bottom wall of the inner cylinder.

15. The multi-dose dispenser of claim 14, wherein the cylinder sprocket has a plurality of teeth that cooperate with a plurality of notches formed on an inner surface of the outer cylinder, the rotation of the inner cylinder relative to the outer cylinder configured to cause corresponding rotations of the sprocket, the expanding plunger base, and the first expanding base of the piston drive system.

16. A multi-dose dispenser for a topical medication, comprising:
a ratchet system permitting a user to selectively provide one of a plurality of measured doses of the topical medication using at least one of a tactile feedback and an acoustic feedback, the ratchet system including an outer cylinder having a switch for selecting one of the plurality of measured doses of the topical medication, and an inner cylinder disposed within of the outer cylinder, the inner cylinder having a plurality of gears that cooperate with the switch to provide the at least one of the tactile feedback and the acoustic feedback during a rotation of the inner cylinder relative to the outer cylinder, the inner cylinder configured to contain the topical medication; and
a piston drive system having a piston disposed within the inner cylinder, the piston drive system including a plurality of expanding bases that are configured to advance the piston along an axial length of the inner cylinder with the rotation of the inner cylinder relative to the outer cylinder, the threaded telescopic piston drive system configured to dispense the topical medication from the inner cylinder, in an amount corresponding to the one of the plurality of measured doses selected, by the rotation of the inner cylinder relative to the outer cylinder, a completion of the rotation confirmed by the at least one of the tactile feedback and the acoustic feedback.

17. The multi-dose dispenser of claim 16, further comprising a main cap disposed on an open end of the inner cylinder, the main cap having at least one hole configured to dispense the topical medication from the inner cylinder.

18. The multi-dose dispenser of claim 17, further comprising an inner cylinder top ring disposed over the outer cylinder and coupled with the inner cylinder, the inner cylinder top ring permitting the rotation of the inner cylinder relative to the outer cylinder.

19. The multi-dose dispenser of claim 18, further comprising a safety lid selectively coupled with the inner cylinder top ring.

20. The multi-dose dispenser in claim 16, further comprising a clear view window formed in the outer cylinder.
